(19) United States
(12) Patent Application Publication
(10) Pub. No.: US 2024/0136073 A1
(43) Pub. Date: Apr. 25, 2024

(54) INFORMATION PROCESSING SYSTEM, INFORMATION PROCESSING METHOD, AND PROGRAM

(71) Applicant: SONY GROUP CORPORATION, TOKYO (JP)

(72) Inventors: TAKAYOSHI HIRAI, TOKYO (JP); NAOYUKI HIROTA, TOKYO (JP); SOICHI FUKASAWA, TOKYO (JP)

(21) Appl. No.: 18/547,764

(22) PCT Filed: Feb. 24, 2022
(86) PCT No.: PCT/JP2022/007465

§ 371 Date: Aug. 23, 2023

(30) Foreign Application Priority Data
Mar. 1, 2021 (JP) 2021-031703

(51) Int. Cl. G16H 50/80

(52) U.S. Cl. CPC G16H 50/80

(57) ABSTRACT

The present technology relates to an information processing system, an information processing method, and a program that enable to hold an event in consideration of infection suppression of infectious diseases. Included are: an acquisition unit configured to acquire ticket information including an attribute of a ticket in an event, user information related to an infectious disease of a user, and event information set by a manager of the event; a determination unit configured to make a determination on the basis of the user information and the setting information; and a setting unit configured to set an attribute of the ticket on the basis of a determination result. The present technology can be applied to, for example, a management server that manages events.

### TECHNICAL FIELD

The present technology relates to an information processing system, an information processing method, and a program.

### BACKGROUND ART

In recent years, measures against infectious diseases have been required in events where people gather, such as live concerts or drama shooting. Thus, various systems have been proposed as measures against infectious diseases (see, for example, Patent Document 1).

### CITATION LIST

### Patent Document

Patent Document 1: Japanese Patent Application Laid-Open No. 2021-7000

### SUMMARY OF THE INVENTION

### Problems to be Solved by the Invention

There is a demand for a system that enables to hold an event in consideration of infection suppression of infectious diseases.

The present technology has been made in view of such a situation, and is intended to further suppress infection of infectious diseases.

### Solutions to Problems

An information processing system according to an aspect of the present technology includes: an acquisition unit configured to acquire ticket information including an attribute of a ticket in an event, user information related to an infectious disease of a user, and event information set by a manager of the event; a determination unit configured to make a determination on the basis of the user information and the setting information; and a setting unit configured to set an attribute of the ticket on the basis of a determination result.

An information processing method according to an aspect of the present technology includes the steps of: acquiring ticket information including an attribute of a ticket in an event, user information related to an infectious disease of a user, and setting information set by a manager of the event by an information processing device configured to manage a ticket management system; making, by the information processing device, a determination on the basis of the user information and the setting information; and setting, by the information processing device, an attribute of the ticket on the basis of a determination result.

A program according to an aspect of the present technology causes a computer to execute the processes of: acquiring ticket information including an attribute of a ticket in an event, user information related to an infectious disease of a user, and setting information set by a manager of the event; making a determination on the basis of the user information and the setting information; and setting an attribute of the ticket on the basis of a determination result.

In an information processing system, an information processing method, and a program according to an aspect of the present technology, ticket information including an attribute of a ticket in an event, user information related to an infectious disease of a user, and setting information set by a manager of the event are acquired, a determination is made on the basis of the user information and the setting information, and an attribute of the ticket is set on the basis of a determination result.

The program can be provided by being transmitted via a transmission medium or by being recorded on a recording medium.

### BRIEF DESCRIPTION OF DRAWINGS

FIG. 1 is a diagram illustrating an outline of a ticket management system implemented by an information processing system according to an embodiment of the present technology.

FIG. 2 is a block diagram illustrating functions of a management server.

FIG. 3 is a block diagram illustrating a configuration example of a management server.

FIG. 4 is a flowchart illustrating an example of registration information processing.

FIG. 5 is a flowchart illustrating a first operation example of a ticket management system.

FIG. 6 is a diagram illustrating an example of zoning of an event venue.

FIG. 7 is a view illustrating an example of a screen displayed on a user terminal.

FIG. 8 is a diagram illustrating an example of grouping of event participating users.

FIG. 9 is a flowchart illustrating a second operation example of a ticket management system.

FIG. 10 is a flowchart illustrating a third operation example of a ticket management system.

FIG. 11 is a diagram illustrating an outline of a mask equipped with a sound collector.

FIG. 12 is a view illustrating an example of an online live concert.

FIG. 13 is a view illustrating an example of a screen displayed on a user terminal.

### MODE FOR CARRYING OUT THE INVENTION

Hereinafter, a mode for carrying out the present technology will be described. The description will be made in the following order.
1. Ticket management system
2. Functions of management server
3. Configuration of management server
4. Example of information registration processing
5. Operation example of ticket management system
6. Mask system
7. Online live concert system

<Ticket Management System>

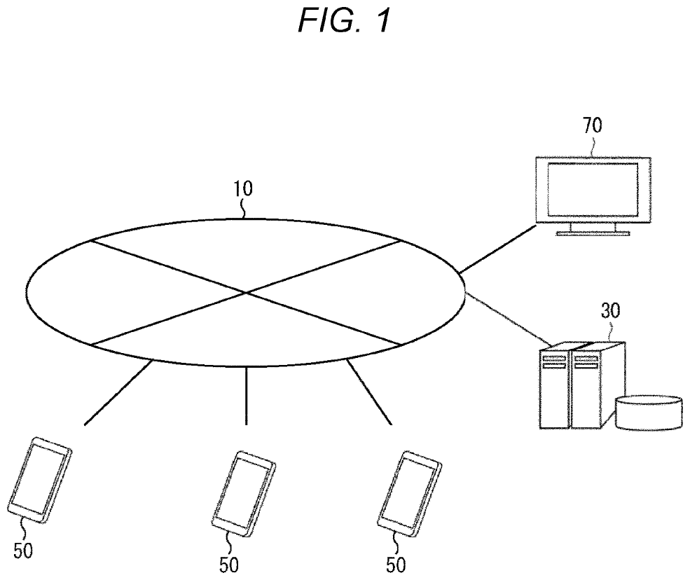

FIG. 1 is a diagram illustrating an example of a ticket management system implemented by an information processing system according to an embodiment of the present technology.

As illustrated in FIG. 1, the ticket management system is a system that manages event tickets using a management server 30. Various functions of the ticket management system are implemented by the management server 30 that is an information processing device connected with a network 10. The management server 30 is configured with one computer or by cooperation of a plurality of computers.

Each user who is an event participant communicates with the management server 30 via the network 10 using an application installed in a user terminal 50 owned by the user. The user terminal 50 is, for example, a smartphone, a personal computer (PC), or the like. A manager who is an event manager controls the management server 30 using a management terminal 70.

Note that the management server 30 and the management terminal 70 may be connected via a network or may be directly connected.

In the ticket management system, the management server 30 changes an attribute of a ticket on the basis of ticket information of an event, user information related to an infectious disease of a user, and setting information set by an event manager. Therefore, the ticket attribute can be changed on the basis of information related to the infectious disease of the user, so that it is possible to hold an event in more consideration of infection suppression. The attribute of the ticket is an attribute indicating whether to allow the user to participate or not and a participation method with the ticket included in the ticket information.

The event is an event in which a plurality of people gathers or an event accessed by a plurality of people, and is, for example, a live concert, drama shooting, an online live concert, or the like. Furthermore, the user is a participant who acquires a ticket and participates in the event. Furthermore, the manager is a person who manages the event, and a plurality of managers may be set. Furthermore, the manager may be a person entrusted by a person who sponsors the event.

<Functions of Management Server>

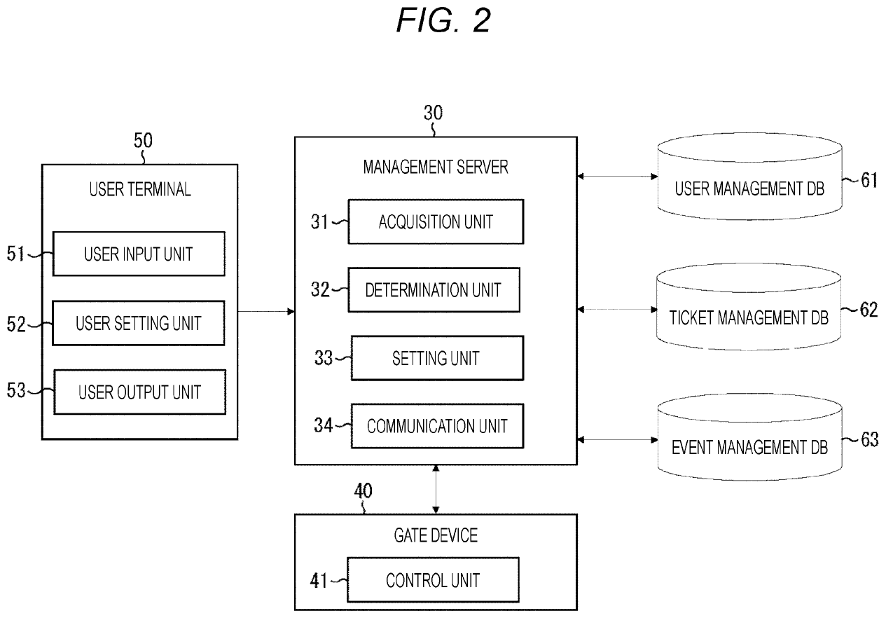

Functions of the management server 30 that implements the ticket management system described above will be described in detail. FIG. 2 is a functional block diagram illustrating functions of the management server 30.

The management server 30 is configured with functions defined by an acquisition unit 31, a determination unit 32, a setting unit 33, and a communication unit 34.

The management server 30 is configured to be able to communicate with a ticket management database (DB) 61, an information management database (DB) 62, an event management database (DB) 63, a gate device 40, and a user terminal 50.

Note that the user management DB 61, the ticket management DB 62, and the event management DB 63 may be DBs stored in an internal memory of the management server 30, or may be DBs stored in a memory of an external device.

Note that information included in each of the user management DB 61, the ticket management DB 62, and the event management DB 63 may be stored as one database. Furthermore, the management server 30 may be configured to be directly connected with the gate device 40 to communicate with gate device 40, or may be configured to communicate with the gate device 40 via a network. Furthermore, the management server 30 may be configured to directly communicate with the user terminal 50 or may be configured to communicate with the user terminal 50 via a network.

The acquisition unit 31 communicates with the user terminal 50, the gate device 40, the user management DB 61, the ticket management DB 62, and the event management DB 63 to acquire information. The information acquired by the acquisition unit 31 is, for example, ticket information of an event, user information related to an infectious disease of a user, setting information set by an event manager, control information of a gate, and the like.

The determination unit 32 makes a determination on the basis of the information acquired by the acquisition unit 31. For example, the determination unit 32 determines whether to allow the user to participate in the event or not and an event participation method on the basis of the user information related to an infectious disease of the user and the setting information set by the event manager.

The setting unit 33 sets a ticket attribute on the basis of a result of determination by the determination unit 32. For example, as setting of the ticket attribute, designation of a gate where the user can enter with the ticket or a viewing position, setting of a viewing group, setting of an online live concert, or the like is performed. That is, information corresponding to a ticket attribute included in ticket information held in the ticket management DB 62 is updated.

Note that the setting unit 33 may set a ticket attribute in an application of the user terminal 50. Furthermore, the setting unit 33 may set a gate condition such as a gate device.

The communication unit 34 communicates with the gate device 40, the user terminal 50, and various DBs to transmit the ticket attribute, control information, and the like set by the setting unit 33.

<Configuration of Management Server>

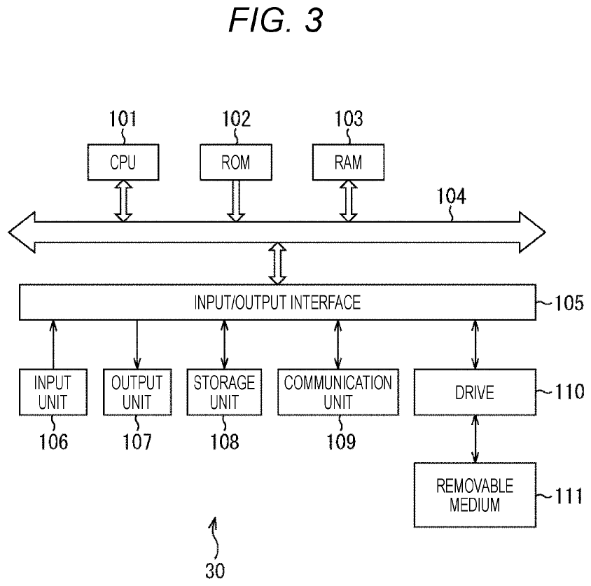

FIG. 3 is a block diagram illustrating a configuration example of the management server 30.

The management server 30 can be configured with a computer. The management server 30 may be configured with a plurality of computers. In a case where the management server 30 is configured with a plurality of computers, the above-described various types of processing are realized by cooperation of the respective computers.

As illustrated in FIG. 3, the management server 30 includes a central processing unit (CPU) 101, a read only memory (ROM) 102, and a random access memory (RAM) 103, which are mutually connected by a bus 104. The bus 104 is further connected with an input/output interface 105. The input/output interface 105 is connected with an input unit 106, an output unit 107, a storage unit 108, a communication unit 109, and a drive 110.

The input unit 106 is configured with a keyboard, a mouse, or the like. The output unit 107 is configured with a display or the like. The storage unit 108 is configured with a hard disk, a nonvolatile memory, or the like. The storage unit 108 stores various types of information such as a program executed by the CPU 101.

The communication unit 109 is an interface for the Internet. For example, the communication unit 109 communicates with the user terminal 50 and various DBs to transmit and receive information to and from the user terminal 50 and the various DBs. Furthermore, the communication unit 109 communicates with the gate device 40 to transmit and receive information to and from the gate device 40. The drive 110 controls writing of data to a removable medium 111 and reading of data from the removable medium 111.

<Example of Information Registration Processing>

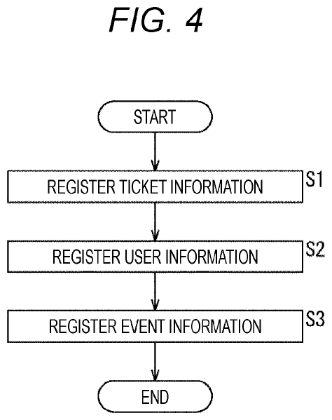

Information registration processing performed in the ticket management system via an application on the user terminal 50 will be described with reference to a flowchart in FIG. 4. FIG. 4 is a flowchart illustrating an example of registration information processing.

In step S1, the management server 30 communicates with the user terminal 50 via the network 10 to acquire ticket information inputted by the user, and stores the ticket information in the ticket management DB 62. A configuration is employed in which the user inputs a user ID and a password to log in to an application installed in the user terminal 50 and inputs the ticket information on the application at this time, so that the management server 30 can acquire the ticket information.

Note that the application installed in the user terminal 50 may be a dedicated application for ticket management or may be a browser application. These applications will be hereinafter referred to as ticket applications.

The ticket information is information regarding event participation. The ticket information is information including, for example, a ticket ID, a user ID associated with a ticket, an event in which a user can participate with a ticket, participation arrangement of an event, information on an entrance/exit gate used for participation in an event, whether to allow the user to participate in an online event or not, and the like.

The user ID is information for identifying a user, and is, for example, an ID used when the user purchases a ticket. The ticket information may be registered in the management server 30 when the user purchases a ticket on a ticket application, or may be registered in the management server 30 by registering a ticket on a ticket application using a serial number or a QR code (registered trademark) of a previously purchased ticket.

Note that the user performs inputting, setting, and outputting of a ticket application or the like by using functions defined as a user input unit 51 (FIG. 2) that is an input function of the user terminal 50, a user setting unit 52 that is a setting function of the user terminal 50, and a user output unit 53 that is an output function of the user terminal 50.

In step S2, the management server 30 acquires the user information by communicating with the user terminal 50, testing equipment (not illustrated), or the like via the network 10, and stores the user information in the user management DB 61.

The user information is information related to an infectious disease of a user, and includes vaccination information indicating a vaccination state, test information indicating a test result of an infectious disease test such as a PCR test, a behavior history of the user, or the like.

For example, when the user activates the ticket application on the user terminal 50 and images a vaccination certificate indicating the vaccination state with a camera, the ticket application transmits the captured image to the management server 30. The management server 30 analyzes the captured image to determine the vaccination state, and stores the determination result in the user management DB 61 as user information. At this time, the user ID and the vaccination information are stored in association with the user information.

Note that the user may register the vaccination information by transmitting a digital certificate indicating the vaccination state to the management server 30 on the ticket application. Furthermore, in a case where the vaccination state is registered in association with the Individual Number (national identification number), the user may read an IC chip of his/her own Individual Number Card using the user terminal 50 or an IC card reader (not illustrated), and transmit the acquired electronic certificate to the management server 30 on the ticket application to register the vaccination information. With this arrangement, the management server 30 can check the validity of the electronic certificate using an online qualification confirmation system in which the electronic certificate is managed by the country or the local government and, in a case where the electronic certificate is valid, acquire the vaccination information of the user from the server of the qualification confirmation organization and store the acquired vaccination information in the user management DB 61.

Regarding the test information, the user may input a test result by himself/herself on the ticket application, or may input a test number, a QR code (registered trademark) outputted from testing equipment, or the like. Furthermore, the user terminal 50 may communicate with testing equipment that tests an infectious disease to acquire a test result, and the test result may be registered in a ticket application of the user terminal 50 without bothering the user.

For example, when a test of an infectious disease is carried out at an event venue and a QR code (registered trademark) generated by a ticket application of the user terminal 50 is held over a test reception terminal, the user ID is stored in the test reception terminal. The test reception terminal issues a test ID associated with the user ID, and stores the result of the test by the testing equipment (for example, PCR testing equipment) in association with the test ID and the user ID. The test reception terminal transmits the test result, the test ID, and the user ID in association with each other to the user terminal 50 or the management server 30.

Note that consent to share the test result to the outside for determination of event participation may be acquired from the user at the time of test reception, and the test reception terminal may transmit the test result to the management server 30 or the user terminal 50 together with the consent information indicating that the consent has been obtained.

A configuration may be employed in which the test result is stored only in the test reception terminal and the ticket application of the user terminal. That is, determination and setting for setting a ticket attribute to be described later may be performed in the ticket application. With this arrangement, there is no need to transmit personal information such a test result of the user to the management server 30, and protection of personal information can be enhanced. Note that the test information may include information regarding health, such as a body temperature test result, an antibody test result, or specific disease information.

In step S3, the management server 30 acquires the event information inputted via the management terminal 70 and stores the event information in the event management DB 63. The event information is information indicating what kind of event participation method is to be used under what kind of condition. For example, the event information is information indicating that the user participates in a dense space in a front region of the live concert venue if the user has been vaccinated, and the user participates in a non-dense space in a back region of the live concert venue if the user has not been vaccinated. This event information is set by an event manager.

<Operation Example 1 of Ticket Management System>

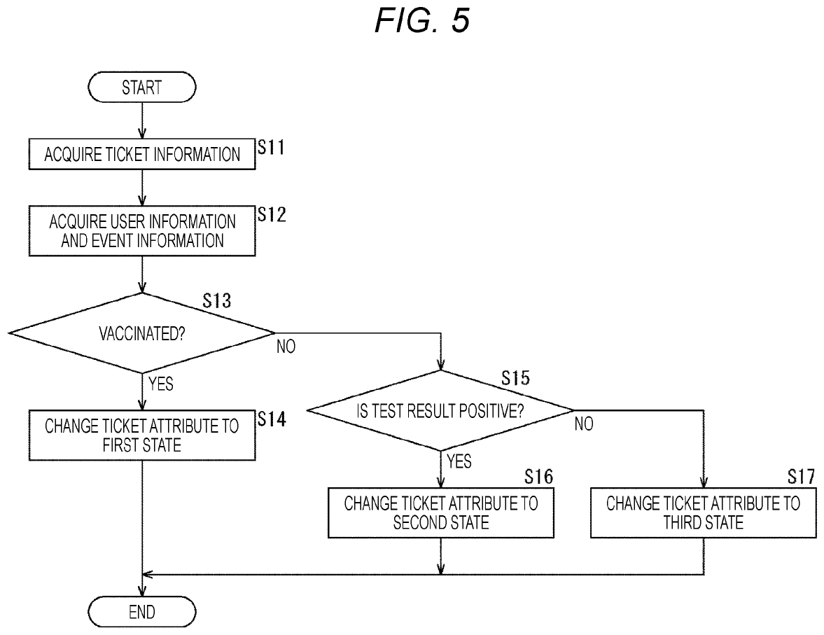

Processing performed by the management server 30 in the ticket management system will be described with reference to a flowchart in FIG. 5. FIG. 5 is a flowchart illustrating a first operation example of the ticket management system.

In step S11, the management server 30 acquires ticket information from the ticket management DB 62.

In step S12, the management server 30 acquires the user information regarding an infectious disease of the user from the user management DB 61. For example, the vaccination information and the test information are acquired as the user information. Furthermore, the management server 30 acquires the event information from the event management DB 63. At this time, the management server 30 may first acquire the event information and then determine the user information to be acquired on the basis of the event information. With this arrangement, it is possible to enhance protection of personal information by avoiding inadvertent acquisition of information unnecessary for determination of an attribute of a ticket.

In step S13, the management server 30 determines whether the user has been vaccinated or not on the basis of the user information. If the user has been vaccinated, the management server 30 advances the processing to step S14, and changes the ticket attribute of the user to a first state. The first state is, for example, a state in which the event participation position is in a dense space in a front region of the event venue.

Note that, at the time of determination, the number of times of vaccination, the number of days of vaccination, the presence or absence of a target vaccine, and the like may also be added to the determination target as the user information.

On the other hand, in a case where it is determined in step S13 that the user has not been vaccinated, the processing is advanced to step S15.

In step S15, the management server 30 determines whether the test result regarding the infectious disease of the user is positive or negative on the basis of the user information. In a case where the result is positive, the management server 30 advances the processing to step S16, and changes the ticket attribute of the user to a second state.

On the other hand, in a case where it is determined in step S15 that the result is negative, the management server 30 advances the processing to step S17, and changes the ticket attribute of the user to a third state.

The second state is, for example, a state in which the user cannot participate at the event venue. The third state is, for example, a state in which the event participation position is in a non-dense space in a back region of the event venue.

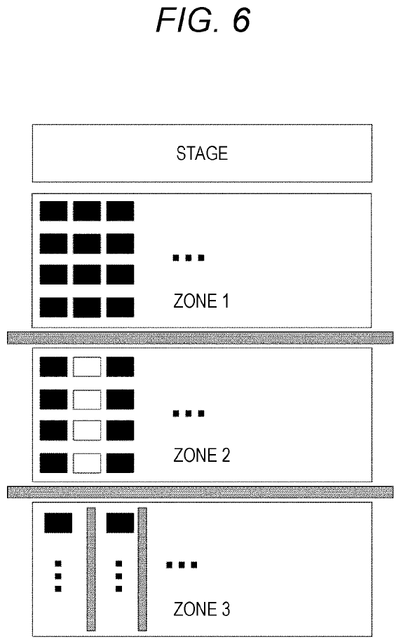

FIG. 6 is a diagram illustrating an example of zoning of an event venue. The physical distance to the stage where the performer is present becomes longer in the order of zone 1, zone 2, and zone 3, and transparent partitions (parts shown in gray in FIG. 6) are disposed between zones. The event participants can be densely arranged in zone 1, while the event participants are arranged at intervals in zone 2. In zone 3, the event participants are arranged at intervals, and furthermore, transparent partitions are also arranged in the zone.

For example, when an event participant who has been vaccinated is set to zone 1, an event participant who has not been vaccinated but got a negative test result on the day is set to zone 2, and an event participant who has not been vaccinated and has not been tested is set to zone 3 as the event information, the management server 30 changes the attribute of the ticket of an event participant on the basis of the user information and the event information. That is, the management server 30 changes the information on the event participation position included in the ticket information of each event participant on the basis of the vaccination result and the test result.

At this time, gates for entering into the respective zones can be installed at different positions. With this arrangement, the number of times of contact between participants belonging to different zones can be reduced. At this time, the management server 30 can control the gate device 40 on the basis of the ticket attribute via a control unit 41 (FIG. 2) that is a control function of the gate device 40. That is, a gate through which the user can enter and exit can be managed on software.

A configuration may be employed in which the ticket application or the management server 30 alerts the user terminal 50 in a case where there is a user in a different zone or gate.

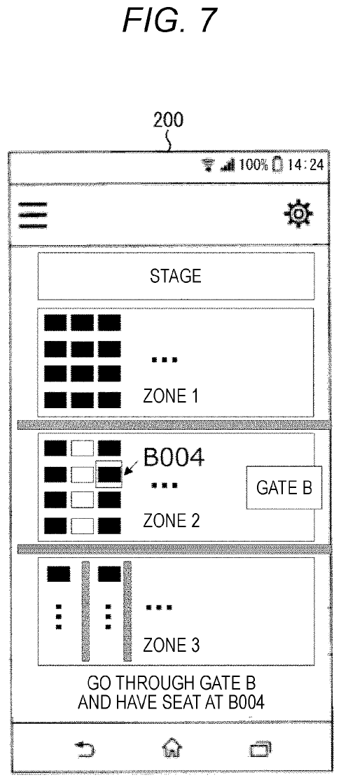

It is also possible to notify the user of the entrance gate or the event participation position on the basis of the ticket attribute set in the ticket application of the user terminal 50. FIG. 7 illustrates an example of a screen displayed on a display 200 of the user terminal 50.

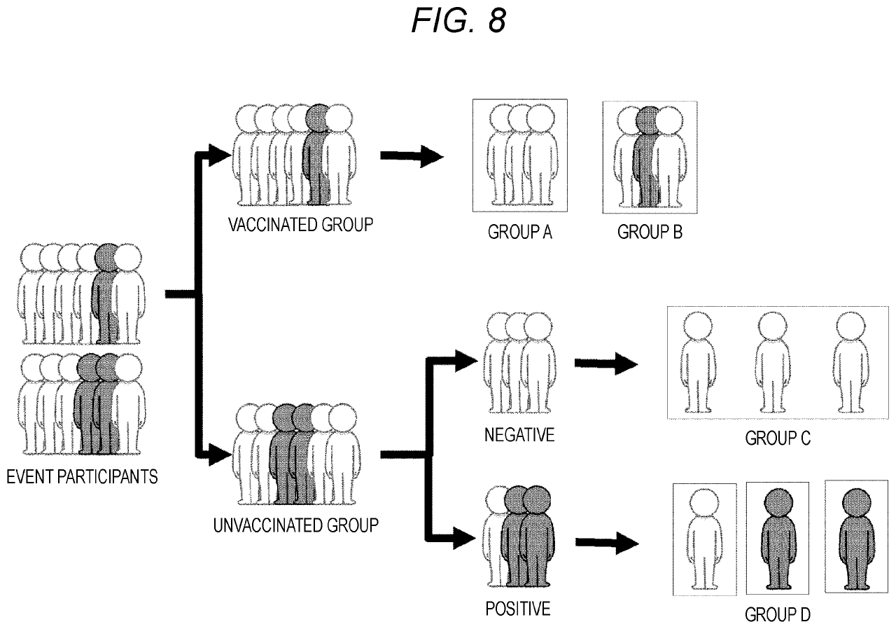

It is also possible to divide each zone into groups, so that the event participants can be arranged at intervals between groups. Moreover, the management server 30 can prompt an event participant to confirm the physical condition every day after event participation. With this arrangement, in a case where there is an event participant who is found after event participation to have been infected with an infectious disease, users belonging to a group in which the infected event participant participated or a neighboring group can be specified from the ticket management DB 62 or the user management DB 61, and a notice of calling attention can be given to the corresponding users. FIG. 8 is a diagram illustrating an example of grouping of event participating users.

Note that the physical condition confirmation sent to each event participant after event participation can be a physical condition confirmation method in which an event benefit is obtained by inputting the physical condition on a ticket application. For example, when the user answers the physical condition on the ticket application of the user terminal 50, the management server 30 sets a bonus video of the event to be browsable on the ticket application of the user terminal 50.

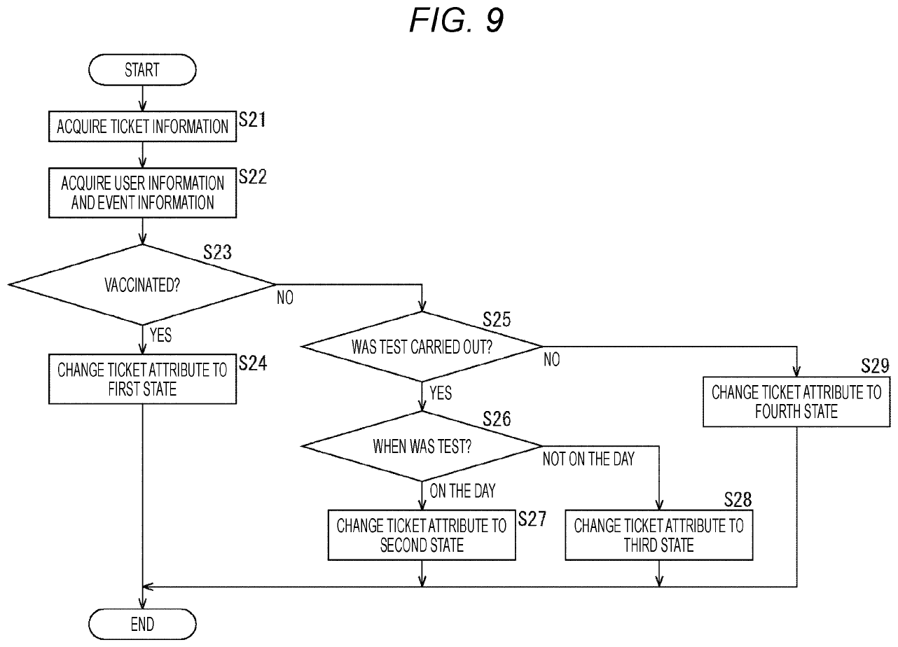

A configuration may be employed in which not only the vaccination information and the test information but also information indicating when the test was carried out is acquired as the user information and the ticket attribute is set. FIG. 9 is a flowchart illustrating a second operation example of the ticket management system.

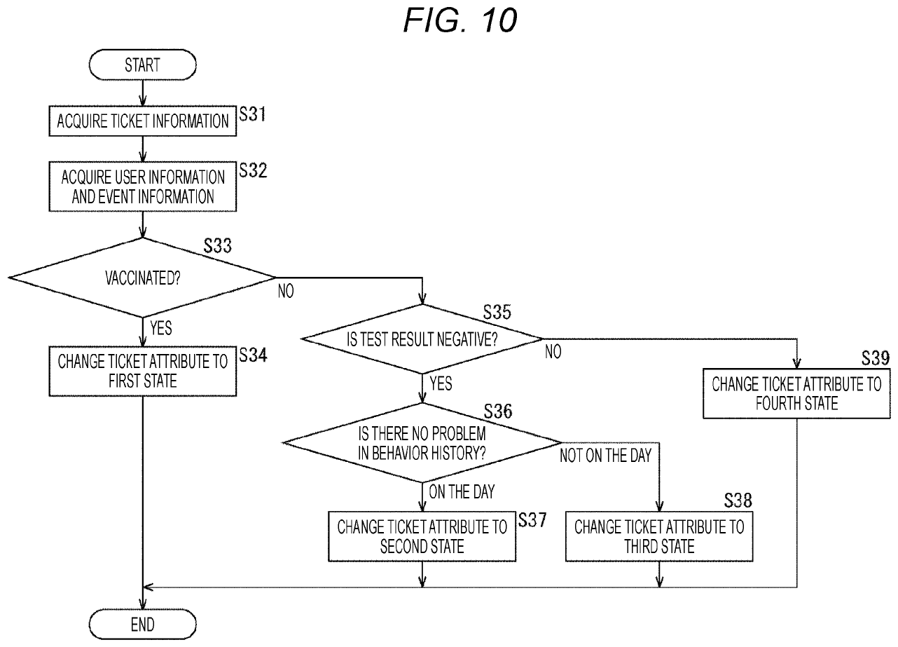

A case of employing a configuration in which not only the vaccination information and the test information but also the behavior history of the user is acquired as the user information and the ticket attribute is set will be described. FIG. 10 is a flowchart illustrating a third operation example of the ticket management system.

According to the third operation example, in a case where there is a user who has infectivity due to the viral load in his/her body, a false negative, or the like but has got a negative test result, it is determined that the risk is low as long as there is no problem in the behavior history, and the ticket attribute can be changed.

The behavior history is, for example, GPS information. The ticket application acquires GPS information to determine whether an infected person has been found in the action range of the user or not or whether the user has stopped by at a high infection risk area such as a tavern or not, thereby determining whether there is a risk or not. Furthermore, instead of the behavior history information of the user, health management history information of the user may be used as the user information. For example, information on the user such as a body temperature, a heart rate, a breathing rhythm, or a sleep rhythm measured every day may be used as the user information to set the ticket attribute.

The ticket attribute can also include whether to allow a user to browse an online live concert or not, or browsing conditions. Furthermore, whether to allow a user to browse an online live concert or not, a period during which the online live concert is browsable, the image quality of the online live concert, and a bonus video may be different depending on the ticket attribute. For example, the management server 30 changes the ticket attribute so that the online live concert becomes browsable at the event start time in a case where the test result is positive, while the online live concert becomes browsable after the event finish time in a case where the test result is negative.

Furthermore, in a case where the test result is positive, a benefit such as an online conversation time with the event performer may be granted. Furthermore, the management server 30 may change the ticket attribute so that, although all the ticket purchasers can browse the online live concert, the vaccinated event participants and the event participants whose test result was positive can browse the online live concert with high image quality even after the event finish time, while the other event participants can browse the online live concert with high image quality only from the event start time to the event finish time and can browse the online live concert only with low image quality after the event finish time. With this arrangement, it is possible to give favorable treatment to or follow the vaccinated event participants or the event participants whose test result was positive while allowing event participants who cannot participate at the venue due to the infection risk to participate in the online live concert.

<Mask System>

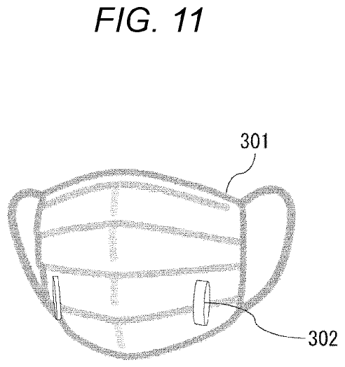

A configuration may be employed in which the user participates in the event at the event venue wearing a mask equipped with a sound collector illustrated in FIG. 11, and the management server 30 changes the performance on the basis of the sound of the user. A sound collector 302 includes a suction unit, a sound collection unit, and a wireless unit. The sound collector 302 is configured to be attachable to a mask 301 by the suction unit. Note that the sound collector and the mask 301 may be integrated.

The sound collection unit extracts the volume of the voice of the user, and transmits the information as cheer information to the management server 30 via the wireless unit. Note that a configuration may be employed in which only the volume is acquired, since the conversation content is personal information. The management server 30 may store the cheer information and the event participation position of the user in association with each other, and generate event performance information based on the cheer information and the event participation position. For example, the management server 30 can generate a video based on a difference between the loudness of voice on the right side of the zone 1 and the loudness of voice on the left side of the zone 1, or a statistical video of a cheer state, and display the video or the statistical video at the event venue.

Note that the cheer information may be generated on the basis of the number of times of tapping on a predetermined screen in the application, instead of the voice.

A configuration may be employed in which the cheer information of an online live concert participant is generated by sound input to the microphone of the user terminal 50 or by screen tapping, and transmitted to the management server 30. With this arrangement, the online live concert participant can deliver a cheer to the artist, and the sense of satisfaction obtained from participating in the live concert is improved.

<Online Live Concert System>

A video of the online live concert can be generated by a volumetric imaging system that combines images captured by a plurality of cameras to generate a stereoscopic video. For example, the management server 30 sets the ticket attribute so that only a video of the live concert captured from the front can be viewed in a case where the test result is negative, and sets the ticket attribute so that a video of the live concert generated by the volumetric imaging system can also be viewed in a case where the test result is positive.

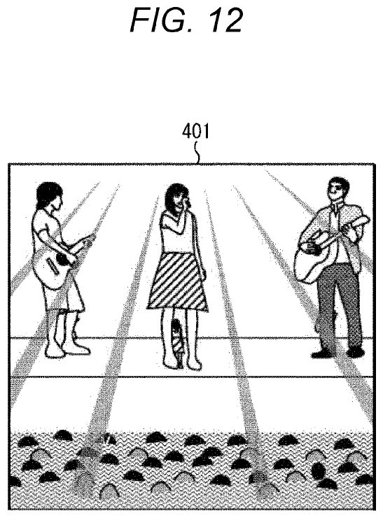

FIG. 12 is a view illustrating an example of an online live concert.

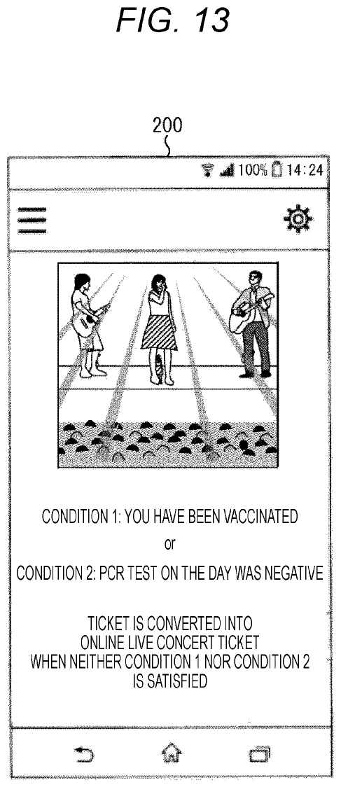

Note that a configuration may be employed in which the ticket application acquires a condition for participating in an event upon purchase of a ticket, and the condition for participating in the event is presented to the user in the ticket application. For example, a screen illustrated in FIG. 13 is presented to the user on the display 200 of the user terminal 50. The user can confirm the condition for participating in the event on the screen displayed in the ticket application. Furthermore, a configuration may be employed in which a test station on the day is displayed in the ticket application.

In which time zone the user can enter the event venue may be displayed in the ticket application, and the management server 30 may control the gate device 40 so that the user cannot enter at a time that is not included in the time zone. For example, the management server 30 may cause a ticket application of the user terminal 50 to display a message of “Gather at PCR test station at 13:00-13:10”, and a control may be performed so that the gate is not opened with a ticket associated with the ticket application except for the time zone.

Moreover, the management server 30 may designate a place or a time for waiting for a test result to the user via the ticket application. Furthermore, a configuration may be employed in which a bonus video of the event is viewable from the ticket application in a state where the participant is waiting for a test. With this arrangement, the user can be entertained even in a case where the waiting time becomes long.

A configuration that prompts the user to wait using the position information of the user may be added. For example, the management server 30 may set the bonus video of the event to be browsable by the user on the basis of the position information and the test information of the user when the user goes to the place designated by the ticket application after being tested, and set the bonus video to be unbrowsable when the user is away from the designated position by a predetermined distance or more. With this arrangement, it is possible to prevent a user, who has not got the test result yet and may be positive, from moving excessively during the waiting time.

<Compatibility with 5G>

The ticket application and the mask system described above may be implemented by using a 5G network. In some cases, several thousands to several tens of thousands of people gather at an event venue, and there is a possibility that all users cannot be simultaneously connected with a network in the 4G network. Thus, the 5G network may be used so that the management server 30 communicates with the ticket application and the mask system.

In particular, edge computing proposed as multi-access edge computing (MEC) may be used. That is, each user terminal 50 may be configured as an edge computer to execute some of the functions of the ticket management system on each user terminal 50. With this arrangement, in addition to the speed improvement by the distributed processing, it is also possible to enhance personal information protection by, for example, limiting provision of personal information only to the user terminal 50.

<Regarding Program>

The series of processing described above can be executed by hardware or by software. In a case where the series of processing is executed by software, a program constituting the software is installed to a computer incorporated in dedicated hardware, a general-purpose personal computer, or the like.

The program to be installed is provided by being recorded in the removable medium 111 illustrated in FIG. 3 configured with an optical disk (a compact disc-read only memory (CD-ROM), a digital versatile disc (DVD), or the like), a semiconductor memory, or the like. Furthermore, the program may be provided via a wired or wireless transmission medium such as a local area network, the Internet, or digital broadcasting. The program can be installed in advance in the ROM 102 or the storage unit 108.

The program executed by a computer may be a program in which processes are performed in time series in the order described in the present specification, or may be a program in which processes are performed in parallel or at necessary timing such as when a call is made or the like.

In the present specification, the system means a set of a plurality of components (devices, modules (parts), and the like), and it does not matter whether all the components are located in the same housing or not. Accordingly, both a plurality of devices housed in separate housings and connected via a network, and one device having a plurality of modules housed in one housing are systems.

Note that the effects described in the present specification are merely illustrative and not restrictive, and other effects may be provided.

Embodiments of the present technology are not limited to the above-described embodiments, and various modifications can be made without departing from the gist of the present technology.

For example, the present technology can have a configuration of cloud computing in which functions of the management server 30 are shared and processed in cooperation by a plurality of devices via a network.

Furthermore, each of the steps described in the above-described flowcharts can be executed by one device, or can be executed in a shared manner by a plurality of devices.

Moreover, in a case where a plurality of processes is included in one step, the plurality of processes included in one step can be executed by one device, or can be executed in a shared manner by a plurality of devices.

<Example of Combination of Configurations>

The present technology can have the following configurations.

(1)

An information processing system including:

an acquisition unit configured to acquire ticket information including an attribute of a ticket in an event, user information related to an infectious disease of a user, and setting information set by a manager of the event;

a determination unit configured to make a determination on the basis of the user information and the setting information; and

a setting unit configured to set an attribute of the ticket on the basis of a determination result.

(2)

The information processing system according to (1),

in which the user information includes a vaccination result regarding an infectious disease of the user,

the determination unit determines whether the vaccination result is valid or not, and

the setting unit sets an attribute of the ticket to a first attribute in a case where the vaccination result is valid, and sets an attribute of the ticket to a second attribute in a case where the vaccination result is not valid.

(3)

The information processing system according to (2),

in which the user information includes a test result regarding an infectious disease of the user,

the determination unit makes a determination on the basis of a test result of a test regarding an infectious disease of the user in a case where the vaccination result is not valid, and

the setting unit sets an attribute of the ticket to a third attribute in a case where the test result is positive, and sets an attribute of the ticket to a fourth attribute in a case where the test result is negative.

(4)

The information processing system according to (3),

in which the acquisition unit acquires behavior history information of the user, and

the determination unit further changes an attribute of the ticket on the basis of the behavior history information in a case where the test result is negative.

(5)

The information processing system according to (3),

in which the acquisition unit acquires timing information indicating a timing at which a test is carried out as the user information,

the determination unit makes a determination on the basis of the test result and the timing information, and

the setting unit changes an attribute of the ticket on the basis of the determination result.

(6)

The information processing system according to any one of (1) to (5),

in which the determination unit determines whether to allow the user to participate in the event or not on the basis of the user information and the setting information.

(7)

The information processing system according to any one of (1) to (6),

in which the determination unit determines whether to allow the user to participate in an online live concert or not on the basis of the user information and the setting information.

(8)

The information processing system according to any one of (1) to (7),

in which the determination unit determines whether to grant an online benefit or not on the basis of the user information and the setting information.

(9)

The information processing system according to any one of (1) to (8),

in which the determination unit determines a condition of an online live concert on the basis of the user information and the setting information, and

the setting unit changes a setting of an online live concert that can be viewed with the ticket on the basis of the determination result.

(10)

An information processing method including the steps of:

acquiring ticket information including an attribute of a ticket in an event, user information related to an infectious disease of a user, and setting information set by a manager of the event by an information processing device configured to manage a ticket management system;

making, by the information processing device, a determination on the basis of the user information and the setting information; and

setting, by the information processing device, an attribute of the ticket on the basis of a determination result.

(11)

A program for causing a computer to execute the processes of:

acquiring ticket information including an attribute of a ticket in an event, user information related to an infectious disease of a user, and setting information set by a manager of the event;

making a determination on the basis of the user information and the setting information; and

setting an attribute of the ticket on the basis of a determination result.

### REFERENCE SIGNS LIST

30 Management server
31 Acquisition unit
32 Determination unit
33 Setting unit
34 Communication unit
40 Gate device
50 User terminal
61 User management DB
61 Ticket management DB
63 Event management DB

1. An information processing system comprising:
an acquisition unit configured to acquire ticket information including an attribute of a ticket in an event, user information related to an infectious disease of a user, and setting information set by a manager of the event;
a determination unit configured to make a determination on a basis of the user information and the setting information; and
a setting unit configured to set an attribute of the ticket on a basis of a determination result.

2. The information processing system according to claim 1,
wherein the user information includes a vaccination result regarding an infectious disease of the user,
the determination unit determines whether the vaccination result is valid or not, and
the setting unit sets an attribute of the ticket to a first attribute in a case where the vaccination result is valid, and sets an attribute of the ticket to a second attribute in a case where the vaccination result is not valid.

3. The information processing system according to claim 2,
wherein the user information includes a test result regarding an infectious disease of the user,
the determination unit makes a determination on a basis of a test result of a test regarding an infectious disease of the user in a case where the vaccination result is not valid, and
the setting unit sets an attribute of the ticket to a third attribute in a case where the test result is positive, and sets an attribute of the ticket to a fourth attribute in a case where the test result is negative.

4. The information processing system according to claim 3,
wherein the acquisition unit acquires behavior history information of the user, and
the determination unit further changes an attribute of the ticket on a basis of the behavior history information in a case where the test result is negative.

5. The information processing system according to claim 3,
wherein the acquisition unit acquires timing information indicating a timing at which a test is carried out as the user information,
the determination unit makes a determination on a basis of the test result and the timing information, and
the setting unit changes an attribute of the ticket on a basis of the determination result.

6. The information processing system according to claim 1,
wherein the determination unit determines whether to allow the user to participate in the event or not on a basis of the user information and the setting information.

7. The information processing system according to claim 1,
wherein the determination unit determines whether to allow the user to participate in an online live concert or not on a basis of the user information and the setting information.

8. The information processing system according to claim 1,
wherein the determination unit determines whether to grant an online benefit or not on a basis of the user information and the setting information.

9. The information processing system according to claim 1,
wherein the determination unit determines a condition of an online live concert on a basis of the user information and the setting information, and
the setting unit changes a setting of an online live concert that can be viewed with the ticket on a basis of the determination result.

10. An information processing method comprising the steps of:
acquiring ticket information including an attribute of a ticket in an event, user information related to an infectious disease of a user, and setting information set by a manager of the event by an information processing device configured to manage a ticket management system;
making, by the information processing device, a determination on a basis of the user information and the setting information; and
setting, by the information processing device, an attribute of the ticket on a basis of a determination result.

11. A program for causing a computer to execute the processes of:
acquiring ticket information including an attribute of a ticket in an event, user information related to an infectious disease of a user, and setting information set by a manager of the event;
making a determination on a basis of the user information and the setting information; and
setting an attribute of the ticket on a basis of a determination result.
